(19) United States
(12) Patent Application Publication
(10) Pub. No.: US 2010/0173865 A1
(43) Pub. Date: Jul. 8, 2010

(54) Anti-Cancer Agent-Hyaluronic Acid Conjugate Compositions and Methods

(76) Inventors: Jim Klostergaard, Kingwood, TX (US); David Farquhar, Houston, TX (US); Sukhen C. Ghosh, Houston, TX (US); Roger Price, Houston, TX (US); Vikas Kundra, Missouri City, TX (US); Ralph S. Friedman, Houston, TX (US)

Correspondence Address: Nielsen IP Law LLC, 1177 West Loop South, Suite 1600, Houston, TX, 77027, US

(21) Appl. No.: 12/605,484

(22) Filed: Oct. 26, 2009

Related U.S. Application Data
(63) Continuation of application No. PCT/US2008/061601, filed on Apr. 25, 2008.
(60) Provisional application No. 60/913,986, filed on Apr. 25, 2007.

(51) Int. Cl. A61K 31/728; C08B 37/08; A61P 35/00

(52) U.S. Cl. 514/54; 536/55.1

(57) ABSTRACT

Methods of making conjugates comprising an anti-cancer agent and hyaluronic acid, together with mixtures of reaction products comprising such conjugates and methods of using such conjugates in therapeutic and research applications are disclosed.

### CROSS-REFERENCE TO RELATED APPLICATIONS

This application is a continuation of International Application No. PCT/US2008/061601 filed Apr. 25, 2008 which claims priority to U.S. Patent. App. Ser. No. 60/913,986 filed Apr. 25, 2007, both of which are incorporated herein by reference.

### STATEMENT REGARDING FEDERALLY SPONSORED RESEARCH OR DEVELOPMENT

This disclosure was developed at least in part using funding from the DOD Ovarian Cancer Research Program, Grant No. DAMD17-00-1-0726. The U.S. government may have certain rights in this invention.

### BACKGROUND

Numerous human tumor types, including ovarian cancer, breast cancer, non-small cell lung cancer, colorectal cancer, head and neck cancers, and other malignancies, have a significant expression of the CD44 family of cell-surface proteoglycans. For example, the CD44 proteoglycan family is expressed in as many as about 90% of fresh samples from primary human ovarian tumors or peritoneal implants. Additionally, studies with squamous cell carcinomas of the head and neck have shown up to 75% to have expression of CD44. Typically, epithelial cancer stem cells also express CD44.

The CD44 proteoglycan family includes a parental form and 10 or more isoforms that are major receptors for hyaluronic acid (also referred to herein as “HA”). Hyaluronic acid comprises repeating disaccharide units which are comprised of glucuronic acid and N-acetyl glucosamine. Hyaluronic acid serves a variety of functions within the extracellular matrix, including direct receptor-mediated effects on cell behavior. These effects occur via intracellular signaling pathways in which hyaluronic acid binds to, and is internalized by, CD44 cell surface receptors.

Paclitaxel is a mitotic inhibitor commonly used in cancer chemotherapy. Macromolecular conjugates of paclitaxel have previously been developed as a method to improve drug delivery to a tumor while reducing systemic toxicity. In vivo, polyglutamic acid-paclitaxel conjugates (PGA-paclitaxel; XYOTAX™) have shown increased tumor accumulation of the drug, decreased tumor growth and reduced toxicity as compared to paclitaxel alone. However, it is believed that cellular uptake of PGA-paclitaxel is likely restricted to uptake by fluid-phase pinocytosis. Thus, a conjugate that could exploit the selectivity and efficiency of receptor-mediated uptake might demonstrate even greater improvements in toxicity/efficacy parameters.

### SUMMARY

Methods of making conjugates comprising an anti-cancer agent and hyaluronic acid, together with mixtures of reaction products comprising such conjugates and methods of using such conjugates in therapeutic and research applications are disclosed.

In some embodiments, the methods of making the anti-cancer agent-hyaluronic acid conjugates include coupling an anti-cancer agent with a hyaluronic acid at a pH between about 7.5 to 9.0. In some embodiments, the anti-cancer agent may be conjugated to less than 10 percent of the disaccharide units of the hyaluronic acid.

Prodrug formulations of anti-cancer agent-hyaluronic acid conjugates are also disclosed.

Methods of determining CD44 receptor selectivity of a prodrug are further described herein. Such methods comprise administering to a subject in need thereof a therapeutically effective amount of an anti-cancer agent-hyaluronic prodrug in combination with free hyaluronic acid.

In addition, methods of treating a cancer and/or reducing or eliminating tumor growth rate in a subject in need thereof are described. The methods may comprise administering a therapeutically effective amount of an anti-cancer agent-hyaluronic acid conjugate to the subject, wherein said conjugate is made by coupling an anti-cancer agent to hyaluronic acid at a pH between about 7.5 to 9.0. In some embodiments, the anti-cancer agent may be conjugated to less than 10 percent of the disaccharide units of the hyaluronic acid so that the anti-cancer agent does not interfere with binding of the hyaluronic acid to CD44.

A particular aspect of the present disclosure describes a mixture comprising at least 10 percent of an anti-cancer agent-hyaluronic acid conjugate wherein said mixture was made by combining an N-hydroxysuccinimide ester of a taxane and a hyaluronic acid at a pH between about 7.5 to 9.0.

### DRAWINGS

For a more complete understanding of the present disclosure, and the advantages thereof, reference is now made to the following descriptions taken in conjunction with the accompanying drawings, in which:

FIG. 1A depicts a T₂-weighted coronal MR image of the abdomen of an NMP-1 implanted nude mouse 199 days following tumor inoculation that was treated with a single intraperitoneal injection of 200 mg/kg HA-TXL, 7 days post tumor inoculation. No tumors were observed: compare to Day 28 images of NMP-1 control mice in FIG. 3A.

FIG. 1B depicts a T₂-weighted coronal MR image of the abdomen of an NMP-1 implanted nude mouse 199 days following tumor inoculation that was treated with a single intraperitoneal injection of 200 mg/kg HA-TXL, 7 days post tumor inoculation. No tumors were observed: compare to Day 28 images of NMP-1 control mice in FIG. 3A.

FIG. 2 is a Kaplan-Meyer survival plot of NMP-1-implanted mice treated intraperitoneally either with saline (controls), with 10 or 15 mg/kg Taxol on regimens of every 7 days×3 beginning on Day 7 post tumor implantation, or with a single injection on Day 7 of 180 mg/kg HA-TXL (paclitaxel equivalents). T/C values were 105 and 120 for the 10 and 15 mg/kg multiple-dose Taxol groups, respectively, and 140 for the single dose HA-TXL group (p=0.004 vs. controls by Mantel-Cox).

FIG. 3A shows representative Day 28 T₂-weighted coronal abdominal MR images of: NMP-1-implanted control mice that were sham-treated with saline; arrows indicate examples of tumor masses throughout the abdomen; note the heavy tumor burden and areas of high signal intensity indicating ascites. B=bladder.

FIG. 3B shows representative Day 28 T₂-weighted coronal abdominal MR images of NMP-1-implanted mice that were treated with a multiple dose intraperitoneal injection regimen of 10 mg/kg Taxol; arrows indicate examples of tumor masses throughout the abdomen; note evidence for ascites.

FIG. 3C shows representative Day 28 T₂-weighted coronal abdominal MR images of NMP-1-implanted mice that were treated with a multiple dose intraperitoneal injection regimen of 15 mg/kg Taxol; note the heavy tumor burden and ascites.

FIG. 3D shows representative Day 28 T₂-weighted coronal abdominal MR images of NMP-1-implanted mice that were treated with a single intraperitoneal injection of HA-TXL; note the comparatively modest tumor burden and few areas of high signal intensity indicating ascites. B=bladder.

FIG. 4A provides representative Day 84 coronal T₂-weighted MR images of the abdomens of SKOV-3ip-implanted mice from the control (Panel A) and the 180 mg/kg HA-TXL treatment groups (Panel B). Arrows indicate examples of intraperitoneal tumors; note greater tumor burden in control vs. treated mice. B=bladder.

FIG. 4B provides a comparison of tumor weights derived from MR images of mice bearing SKOV-3ip tumors (Panel C; p<0.03 by t-test, n=3).

FIG. 5 shows an example of a Taxol-hyaluronic acid conjugate that may be present in a product mixture resulting from certain synthesis methods disclosed herein.

FIG. 6A is a graph depicting the in vitro effects of HA-paclitaxel for the OSC19-luciferase cell line using a MTT assay. HA-paclitaxel showed significant growth inhibitory effects, but with slightly decreased potency as compared to paclitaxel alone for the OSC19-luciferase cell line (IC₅₀ 4.31 nM versus 2.16 nM).

FIG. 6B is a graph depicting the in vitro effects of HA-paclitaxel for the paclitaxel-resistant cell line, HN5, using a MTT assay. In the paclitaxel-resistant cell line, HN5, HA-paclitaxel was growth inhibitory at nanomolar concentration (IC₅₀ 11.77 nM), but had decreased potency as compared to paclitaxel (IC₅₀ 4.58 nM).

FIG. 7A is a graph depicting the blocking effect of excess free hyaluronic acid on a HN5 cell line. Pre-incubation with excess free HA blocked the decrease in cell proliferation induced by HA-paclitaxel. This effect was significant in the HN5 cell line at all concentrations (p<0.01).

FIG. 7B is a graph depicting the blocking effect of excess free hyaluronic acid in the OSC19-luciferase cell line. Pre-incubation with excess free HA blocked the decrease in cell proliferation induced by HA-paclitaxel. In the OSC19-luciferase cell line, blocking was only demonstrated at 500 ng/ml HA-paclitaxel, but not at 100 or 50 ng/ml.

FIG. 8A is an image depicting the uptake of HA-paclitaxel-FITC in vitro.

FIG. 8B is an image depicting the uptake of HA-paclitaxel-FITC in vitro.

FIG. 9A is a graph depicting the anti-tumor efficacy of HA-paclitaxel in xenograft models of oral tongue SCC using three groups: control, intravenous free paclitaxel (“TXL”), and intravenous HA-paclitaxel (“HA-TXL”) in OSC19-luciferase cells. Treatment with free paclitaxel decreased the growth of tumor in OSC19 by 64.2% whereas HA-paclitaxel reduced tumor growth by 90.7% one week after the last treatment (p<0.01).

FIG. 9B is a graph depicting the anti-tumor efficacy of HA-paclitaxel in xenograft models of oral tongue SCC using three groups: control, intravenous free paclitaxel (“TXL”), and intravenous HA-paclitaxel (“HA-TXL”) in HN5 cells. Treatment with free paclitaxel decreased the growth of tumor by 63.8% whereas HA-paclitaxel reduced tumor growth by 86.2% one week after the last treatment (p<0.01).

FIG. 10A is a graph depicting the bioluminescence in orthopic tumor xenograft mice. Treatment with free paclitaxel (“TXL”) and intravenous HA-paclitaxel (“HA-TXL”) caused a significant decrease in bioluminescence. Bioluminescence was reduced by 99.2% in the HA-paclitaxel treated animals and by 86.5% in paclitaxel treated animals as opposed to control (p<0.01) as measured at one week after the last treatment. The HA-paclitaxel treated group had significantly lower bioluminescence compared to the free paclitaxel treated group (p<0.01).

FIG. 10B shows representative images of bioluminescence in orthotopic tumor xenograft mice. Treatment with free paclitaxel (“TXL”) and intravenous HA-paclitaxel (“HA-TXL”) caused a significant decrease in bioluminescence. Bioluminescence was reduced by 99.2% in the HA-paclitaxel treated animals and by 86.5% in paclitaxel treated animals as opposed to control (p<0.01) as measured at one week after the last treatment. The HA-paclitaxel treated group had significantly lower bioluminescence compared to the free paclitaxel treated group (p<0.01).

FIG. 11A is a graph depicting the survival rate of orthotopic nude mice treated with free paclitaxel (“TXL”) and intravenous HA-paclitaxel (“HA-TXL”) in OSC-19 luciferase cells. Treatment with HA-paclitaxel or free paclitaxel resulted in increased survival as compared to control by log-rank test (p<0.001). Median survival time for control, paclitaxel, and HA-paclitaxel was 30, 60, and 79 days for OSC19-luciferase.

FIG. 11B is a graph depicting the survival rate of orthotopic nude mice treated with free paclitaxel (“TXL”) and intravenous HA-paclitaxel (“HA-TXL”) in HN5 cells. Treatment with HA-paclitaxel or free paclitaxel resulted in increased survival as compared to control by log-rank test (p<0.001). Median survival time for control, paclitaxel, and HA-paclitaxel was 26, 40, and 45 days for HN5.

FIG. 12A is a graph depicting the effects of free paclitaxel (“TXL”) and intravenous HA-paclitaxel (“HA-TXL”) on angiogenesis. Treatment with free paclitaxel had no effect on MVD, whereas treatment with HA-paclitaxel significantly reduced MVD (p<0.001).

FIG. 12B shows representative images of CD31 staining as a measure of angiogenesis. Treatment with free paclitaxel had no effect on MVD, whereas treatment with HA-paclitaxel significantly reduced MVD (p<0.001).

### DETAILED DESCRIPTION

The present disclosure provides conjugates comprising an anti-cancer agent and hyaluronic acid useful in the treatment of cancer. The disclosure further provides methods of making conjugates comprising coupling an anti-cancer agent with a hyaluronic acid at a pH between about 7.5 to 9.0. Moreover, the disclosure further provides methods of treating a cancer by administering to a subject in need thereof a therapeutic amount of an anti-cancer agent-hyaluronic acid conjugate. Methods of using such conjugates in therapeutic and research applications are also disclosed. In some embodiments, the conjugation of an anti-cancer agent and hyaluronic acid provides the selectivity and efficiency of receptor-mediated uptake and can offer an improved cancer therapeutic in terms of toxicity/efficacy parameters, among other things. The anti-cancer agent-hyaluronic acid conjugates disclosed herein may be useful in treating any cancer cell having a CD44 receptor.

The conjugates described herein are prepared by a novel final coupling step comprising coupling an anti-cancer agent with a hyaluronic acid at a pH between about 7.5 to 9.0. The conjugates of the present disclosure may provide several benefits in that the use of a hydrophilic hyaluronic backbone may both overcome the limited aqueous solubility of certain anti-cancer agents, such as paclitaxel, without the need for an excipient as in Taxol, as well as allow multiple sites for anti-cancer agent loading onto a single hyaluronic scaffold to be internalized by one or more CD44 molecules. Another advantage may be that cancer cells may have a reduced tendency to develop drug resistance to anti-cancer agent-hyaluronic conjugates, than to unconjugated or free paclitaxel. Moreover, by coupling an anti-cancer agent with a hyaluronic acid at a pH between about 7.5 to 9.0, a yield may be achieved that allows for sufficient production of the conjugate.

The conjugates described herein comprise an anti-cancer agent and hyaluronic acid. As used herein, the term “anti-cancer agent” refers to a compound capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Additionally, as used herein, the term “anti-cancer agent” includes an anti-cancer agent derivative having functional groups by which an anti-cancer agent is bonded to a hyaluronic acid. Similarly, as used herein, the term “hyaluronic acid” also includes hyaluronic acid derivatives, including those hyaluronic acid derivatives that have functional groups through which an anti-cancer agent is bonded to a hyaluronic acid backbone.

In some embodiments, anti-cancer agents suitable for use in the conjugates of the present disclosure comprise a taxane. In general, taxanes typically are diterpenes with antineoplastic properties, such as the inhibition of microtubule function. Examples of suitable taxanes include, but are not limited to, paclitaxel, docetaxel, and derivatives thereof. In one embodiment, a suitable anti-cancer agent may be present as an active ester, such as a N-hydroxysuccinimide ester (“NHS ester”). For example, in one embodiment, a suitable anti-cancer agent may be paclitaxel-N-hydroxysuccinimide ester, also referred to as “paclitaxel-NHS ester” or “Taxol-NHS ester.” In some embodiments, a NHS ester of an anti-cancer agent may be coupled to a hyaluronic acid that is modified with a dihydrazide compound such as adipic dihydrazide. By way of example, a suitable conjugate may comprise a paclitaxel anti-cancer agent coupled to a hyaluronic acid.

Other anti-cancer agents may also be suitable for use in the disclosed conjugates. Anti-cancer agents include, for example, chemotherapy agents (chemotherapy), radiotherapy agents (radiotherapy), immune therapy agents (immunotherapy), genetic therapy agents (gene therapy), hormonal therapy, other biological agents (biotherapy) and/or alternative therapies. A non-exhaustive list of anti-cancer agents which may be suitable for use as an anti-cancer agent in the conjugates disclosed herein may be found in U.S. Pat. No. 7,344,829, column 12, line 43 through column 13, line 4, incorporated herein by reference. In some embodiments, a suitable anti-cancer agent-hyaluronic acid conjugate may have one or more of the same and/or different anti-cancer agents conjugated to hyaluronic acid.

The anti-cancer agent-hyaluronic acid conjugates of the present disclosure are prepared by coupling an anti-cancer agent with hyaluronic acid at a pH between about 7.5 to 9.0. The coupling reaction carried out at a pH between about 7.5 to 9.0 can yield a mixture of reaction products comprising at least 10% of an anti-cancer agent-hyaluronic acid conjugate. In some instances, a buffer system may be used to maintain a coupling reaction pH between 7.5 to 9.0. One exemplary buffer system is a NaHCO₃ buffer having a pH of 8.5. By coupling the anti-cancer agent and hyaluronic acid at a pH between about 7.5 and 9.0, a higher yield of conjugates may be obtained.

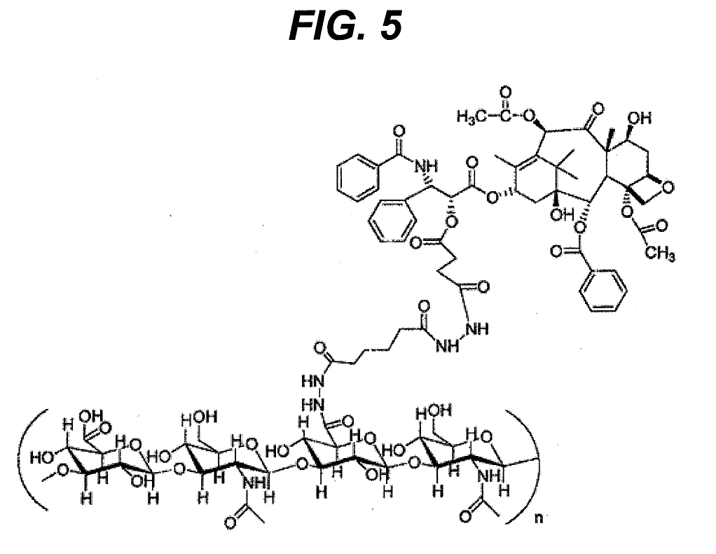

In some embodiments, the anti-cancer agent may be conjugated to the hyaluronic acid so that at least 90% of the disaccharides of the hyaluronic acid backbone are left intact and available for receptor-mediated uptake (e.g., CD44 binding). Accordingly, the anti-cancer agent may be conjugated to less than 10 percent of the disaccharide units of the hyaluronic acid. When the anti-cancer agent is a taxane, the taxane-hyaluronic acid conjugates may contain from about 15-20% taxane (w/w). FIG. 5 illustrates one example of a Taxol—hyaluronic acid conjugate present in a product mixture resulting from certain synthesis methods wherein Taxol-NHS ester is combined with adipic dihydrazido-functionalized hyaluronic acid at a pH between about 7.5 to 9.0.

In some embodiments, anti-cancer agent-hyaluronic acid conjugates of the present disclosure may exist as prodrugs. In general, the term “prodrug” refers to a compound that undergoes a conversion in vivo to an active drug. Certain conjugates of the present disclosure may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). The conjugates described herein may be prodrugs of a compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. The term “therapeutically acceptable prodrug,” refers to those prodrugs which are suitable for use in contact with the tissues of patients without undue toxicity, irritation, and allergic response, are commensurate with a reasonable benefit/risk ratio, and are effective for their intended use.

In another aspect, the present disclosure provides methods for treating cancer-mediated disorders in a human or animal subject in need of such treatment comprising administering to said subject a therapeutically effective amount of an anti-cancer agent-hyaluronic acid conjugate of the present disclosure effective to reduce or prevent said disorder in the subject. The anti-cancer agent-hyaluronic acid conjugates of the present disclosure may be useful in treating any cancer cell having a CD44 receptor. For example, the cancer may be ovarian cancer, breast cancer, non-small cell lung cancer, colorectal cancer, and head and neck cancers.

The phrase “therapeutically effective” is intended to qualify the amount of active ingredients used in the treatment of a disease or disorder. This amount will achieve the goal of reducing or eliminating the said disease or disorder.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

Additionally, methods for reducing or eliminating tumor growth rate in a subject in need thereof are provided. Such methods comprise administering a therapeutically effective amount of an anti-cancer agent-hyaluronic acid conjugate to the subject. The method may further comprise administering additional chemotherapeutic agents.

In certain instances, the conjugates of this disclosure may also be useful in combination with known anti-cancer and cytotoxic agents and treatments such as radiation therapy. Anti-cancer agent-hyaluronic acid conjugates may be used sequentially as part of a chemotherapeutic regimen also involving other anticancer or cytotoxic agents and/or in conjunction with non-chemotherapeutic treatments such as surgery or radiation therapy.

While it may be possible for a conjugate which comprises an anti-cancer agent and hyaluronic acid to be administered as a raw chemical, it is also possible to present such a conjugate as a pharmaceutical formulation. Accordingly, pharmaceutical formulations comprising a conjugate which comprises an anti-cancer agent and hyaluronic acid, together with one or more pharmaceutically acceptable carriers thereof and optionally one more other therapeutic agents, are provided.

The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present disclosure may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association an anti-cancer agent-hyaluronic acid conjugate (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present disclosure may be administered topically, that is by non-systemic administration. This includes the application of a compound of the present disclosure externally to the epidermis or the buccal cavity and the instillation of such a compound into the ear, eye and nose, such that the compound does not significantly enter the blood stream. Additionally, in some embodiments, the compounds of the present disclosure may be administered orally, intravenously, intraperitoneally and intramuscularly. Clinical trial results have provided compelling evidence that intraperitoneal administration of these drugs results in markedly improved survival in small volume disease patients compared to intravenous administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject disclosure may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present disclosure include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present disclosure are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present disclosure may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the compounds according to the disclosure are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the formulations may include other agents conventional in the art having regard to the type of formulation in question, for example those suitable for oral administration may include flavoring agents.

The amount of active ingredient that may be combined with the carrier materials to produce a single dosage form will vary depending upon the host treated and the particular mode of administration.

Besides being useful for human treatment, these compounds are also useful for veterinary treatment of companion animals, exotic animals and farm animals, including mammals, rodents, and the like. More preferred animals include horses, dogs, and cats.

### EXAMPLES

### Example 1

A lead formulation of an anti-cancer agent-hyaluronic acid conjugate, “HA-TXL” was prepared and its toxicity parameters as well as its anti-tumor activity in two CD44(+) human ovarian carcinoma nude mouse xenograft models were evaluated. The results, which establish in vivo characteristics of such an HA-based prodrug, indicate that even a single intraperitoneal administration of a sub-MTD dose of HA-TXL resulted in anti-tumor efficacy: reduced or eliminated tumor burden and prolonged survival compared to controls.

Cell Lines

A cisplatin (CDDP)-resistant cell line was first developed from parental OVCAR-3 cells (American Type Culture Collection, Manassas, Va.) by in vitro incubation with increasing concentrations of CDDP. Kalpna, M., et al., Emergence of CDDP-Resistant Cells from OVCAR-3 Ovarian Carcinoma Cell Line With p53 Mutations, Altered Tumorigenicity and Increased Apoptotic Sensitivity to p53 Gene Replacement, Int J. Gynecol Cancer, 2000, 10:105-114. Cells surviving several rounds of selection in CDDP-containing medium were cloned by limiting dilution, expanded, and retested for CDDP resistance. NMP-1 cells were derived from ascites of nude mice into which these CDDP-resistant OVCAR-3 cells had been implanted intraperitoneally. Auzenne, E., et al., Superior Therapeutic Profile of Poly-L-glutamic Acid-Paclitaxel Copolymer Compared With Taxol in Xenogeneic Compartmental Models of Human Ovarian Carcinoma, Clin Cancer Res, 2002, 8(2): 573-81; Hamilton, T. C., et al., Characterization of a Human Ovarian Carcinoma Cell Line (NIH: OVCAR-3) With Androgen and Estrogen Receptors, Cancer Res, 1983, 43:5379-89.

Synthesis of Taxol-N-hydroxysuccinimide Ester, Adipic Dihydrazido-Functionalized HA, and HA-TXL

Hyaluronic acid (HA, ˜40 kDa) was provided by K3 Corporation (VA, USA). 1-Ethyl-3-[3′-(dimethylamino)propyl]carbodiimide (EDCI), diphenylphosphoryl chloride, adipic dihyrazide (ADH), succinic anhydride, N-hydroxysuccinimide, and triethylamine were purchased from Sigma-Aldrich Company (Milwaukee, Wis.). Paclitaxel (Taxol®) was purchased from HandeTech Development Company (Houston, Tex.). All solvents were of reagent or HPLC grade.

Nuclear magnetic resonance (NMR) spectral data were obtained on a 300 MHz or 500 MHz Bruker Advance Spectrometer. UV-Vis spectra were recorded on a Perkin-Elmer spectrometer. HPLC was carried out on a Waters Model 2695 system equipped with a C-18 column and a 2996 photodiode detector using, as eluent, H₂O—CH₃CN (60:40) as eluent at a flow rate of 1 mL/min.

Synthesis of Taxol-NHS(N-hydroxysuccinimide) Ester: The reported synthesis of Luo and Prestwich was followed. Luo, Y., et al., Synthesis and Selective Cytotoxicity of a Hyaluronic Acid-Antitumor Bioconjugate, Bioconjug Chem, 1999, 10(5):755-63; Luo, Y., et al., A Hyaluronic Acid-Taxol Antitumor Bioconjugate Targeted to Cancer Cells, Biomacromolecules, 2000, 1(2):208-18. To a stirred solution of paclitaxel (540 mg, 0.63 mmol) and succinic anhydride (76 mg, 0.76 mmol) in CH₂Cl₂ (25 mL) at room temperature was added dry pyridine (513 μL, 6.3 mmol). The reaction mixture was stirred for three days at room temperature and then concentrated in vacuo. The residue was dissolved in CH₂Cl₂ (5 mL), and the product was purified by silica gel column chromatography (ethyl acetate-hexane, 1:1) to yield Taxol-2′-hemisuccinate as a white solid (85%).

N-hydroxysuccinimido diphenyl phosphate (SDPP) was prepared from diphenylphosphoryl chloride, N-hydroxysuccinimide, and triethylamine in CH₂Cl₂ as previously described. Luo, Y., et al., Synthesis and Selective Cytotoxicity of a Hyaluronic Acid-Antitumor Bioconjugate, Bioconjug Chem, 1999, 10(5):755-63; Luo, Y., et al., A Hyaluronic Acid-Taxol Antitumor Bioconjugate Targeted to Cancer Cells, Biomacromolecules, 2000, 1(2):208-18. The crude product was triturated with ether, dissolved in ethyl acetate, washed with H₂O, and dried over MgSO₄. Concentration of the organic layer in vacuo gave pure SDPP (85%).

To a solution of Taxol-hemisuccinate (300 mg, 0.31 mmol) and SDPP (164 mg, 0.46 mmol) in acetonitrile (15 mL) was added 175 μL (1.2 mmol) of triethylamine. The reaction was stirred for 6 hours at room temperature, and then concentrated in vacuo. The residue was dissolved in ethyl acetate/hexane and purified by silica gel column chromatography (ethyl acetate-hexene, 1:2). The Taxol-NHS ester was dried for 24 hours in vacuo at room temperature to give 265 mg (80%).

Synthesis of Adipic Dihydrazido-Functionalized HA (HA-ADH): HA-ADH was prepared according to Bulpit and Aeschlimann. Bulpitt, P., et al., New Strategy for Chemical Modification of Hyaluronic Acid: Preparation of Functionalized Derivatives and Their Use in the Formation of Novel Biocompatible Hydrogels, J Biomed Material Res, 1999, 47:152-169. Briefly, HA was dissolved in water to give a concentration of 3 mg/mL. To this solution was added a 30-fold molar excess of ADH. The pH of the reaction mixture was adjusted to 6.8 with 0.1 M NaOH/0.1 M HCl. One equivalent of EDCI was added in solid form followed by 1 equivalent of 1-hydroxybenzotriazole (HOBt) in DMSO-H2O (1:1) solution. The pH of the mixture was maintained at 6.8 by addition of 0.1 M NaOH and the reaction was allowed to proceed overnight. The reaction was quenched by addition of 0.1 N NaOH to pH 7.0. The mixture was then transferred to pretreated dialysis tubing and dialyzed exhaustively against 100 mM NaCl, 25% EtOH/H₂O, and finally H₂O. The solution was filtered through a 0.2 μm cellulose acetate membrane, flash frozen, and lyophilized. The purity of the HA-ADH was determined by HPLC. The extent of substitution of HA with ADH was determined by the ratio of methylene hydrogens to acetyl methyl protons as measured by [¹H]NMR.

Synthesis of HA-TXL: In initial experiments, the method reported by Luo and Prestwich for synthesizing HA-TXL was followed, but low yields of less than about 10% were obtained. Luo, Y., et al., Synthesis and Selective Cytotoxicity of a Hyaluronic Acid-Antitumor Bioconjugate, Bioconjug Chem, 1999, 10(5):755-63; Luo, Y., et al., A Hyaluronic Acid-Taxol Antitumor Bioconjugate Targeted to Cancer Cells, Biomacromolecules, 2000, 1(2):208-18. Those low yields were insufficient to support in vivo studies. As an alternative to Luo and Prestwich's methods, HA-TXL was synthesized as described below, with a major change being a higher pH for final coupling. Using these modified methods, moderate to high yields of at least about 50% were consistently obtained.

In performing the modified methods, HA-ADH (75 mg) was dissolved in 0.1 M NaHCO₃ buffer, pH 8.5, at a concentration of 1 mg/mL. To this solution was added Taxol-NHS ester (18 mg) dissolved in sufficient DMF-H₂O (2:1, v/v) to give a homogeneous solution. The reaction mixture was stirred at room temperature for 24 hours and then evaporated to dryness in vacuo (37° C.). The residue was dissolved in H₂O, and the product was purified by gel filtration chromatography (Biogel P-10; Bio-Rad, Hercules, Calif.) using water as eluent. Fractions containing HA-TXL, as evidenced by HPLC analysis, were combined and lyophilized. The [¹H]NMR spectrum of the product showed phenyl resonances at 7.25 to 8.15 ppm affording proof of the formation of HA-TXL. The purity of the product was determined by HPLC analysis. The percentage of incorporated paclitaxel was determined by UV absorbance (Taxol: λmax=227 nm, ε=2.8×104). In this manner, conjugates with up to about 10% of the carboxyl groups modified were prepared; this level of substitution would leave about 90% or more of the disaccharides intact and available for CD44 binding and produce conjugates containing about 15 to 20% paclitaxel (w/w). For in vitro and in vivo studies, paclitaxel equivalents in terms of concentration and mass, respectively, were calculated for each batch of HA-TXL prepared.

In Vitro Cytotoxicity Assays

NMP-1 and SKOV-3ip cells (1×10⁴ cells/well) were cultured overnight in 96-well plates in 100 μl of medium (Dulbecco's modified Eagle's medium/F12; Life Technologies, Inc.) supplemented with 5% fetal calf serum/well before treatment. The cytotoxic effects of HA-TXL were established using a dose range of drug up to 4 μg/ml (paclitaxel equivalents). Remaining viable cells were stained with neutral red after up to 96 hours, and the percentage of control cell survival as measured by optical density of incorporated dye was determined. In competition studies, cells were pre-treated with a 100-fold molar excess of free HA before 4 hours of incubation with HA-TXL; free HA and HA-TXL were washed off the plate and fresh media added for the rest of the 72-hour incubation period.

In Vivo Efficacy Assays

NMP-1: These studies were designed to give quantitative survival data as criteria for the anti-tumor efficacy of HA-TXL and for its comparison to Taxol. On Day 0, about 1×10⁷ viable NMP-1 cells were injected into the peritoneal cavities of groups of 6 to 9-week-old female nude mice (Harlan Sprague Dawley, Indianapolis, Ind.). Five or more mice per experimental group were used as the basis for statistical analyses. Administration of drugs was initiated 1 week later (Day 7). Complete necropsy and histopathologic evaluation, as well as MR imaging analysis, of mice in parallel studies indicated that within 7 days of intraperitoneal innoculation, abdominal tumors were already present. Taxol was administered intraperitoneally on a schedule of every 7 days×3, at either 10 or 15 mg/kg; higher doses than this frequently resulted in marked toxicity and/or death in hand. HA-TXL (14% paclitaxel by weight) was administered in a single intraperitoneal dose of up to 300 mg/kg in pilot studies and 180 mg/kg of HA-TXL (18% paclitaxel by weight) was used in the main study, the same dose that had previously been used in pre-clinical ovarian carcinoma xenograft studies with PGA-TXL. NMP-1-implanted mice develop marked ascites as one of the earliest clinical signs of peritoneal tumor and before other aspects of tumor progression are apparent; ascitic fluid was repeatedly removed at intervals from mice, beginning around the fourth week. Eventually cachexia, spine prominence, and other morbid symptoms became more severe, and these animals were humanely sacrificed by carbon dioxide asphyxiation. For any tumor-bearing mice that succumbed between daily observations and before the opportunity to sacrifice them, the day of death was considered to be the day before the date they were discovered as deceased. The day of humane sacrifice/death was recorded for each mouse, and these values were compared among control and treatment groups by paired or unpaired Student's t-tests for the survival analyses.

SKOV-3ip: These studies were conducted similarly to those described for the NMP-1 model, except that the mice were subjected to magnetic resonance (MR) imaging-based quantification of remaining tumor volumes at a common endpoint, rather than being taken to a survival endpoint. Further, 1×10⁶ to 2×10⁶ cells were injected intraperitoneally and treatment with HA-TXL was not initiated until Day 14.

Magnetic Resonance Imaging (MRI) Analyses

MRI studies were conducted in the MDACC Small Animal Imaging Facility (SAIF). Previous studies revealed that these orthotopic intraperitoneal human ovarian carcinoma xenograft models initially presented either as numerous widely dispersed foci of individual and coalescing solid tumors throughout the peritoneal cavity or as more solid masses which appeared to originate adjacent to and around the pancreas. Klostergaard, J., et al., Magnetic Resonance Imaging-Based Prospective Detection of Intraperitoneal Human Ovarian Carcinoma Xenografts Treatment Response, Int J Gynecol Cancer, 2006, 16 Suppl 1:111-7. Respiratory-gated, T₂-weighted (T_{E}: 45.0 ms, T_{R}: 1215.6 ms, 0.5 mm thickness, 0.3 mm space between images) coronal images were used for initial evaluation of tumor distribution and growth in these models; images of the abdomens of these mice were acquired using a Bruker 4.7 T, 40 cm Biospec MR scanner (Bruker Biospin USA, Billerica, Mass.). Preliminary studies had demonstrated that peritoneal tumors as small as 500 microns in diameter were detectable; generally, MR imaging-based evidence of tumor was first clearly detected on Day 7 (NMP-1) and Day 14 (SKOV-3).

In the NMP-1 studies, mice were held for survival endpoints. In the SKOV-3ip studies, tumor measurements were performed using the Image J program (National Institutes of Health, USA). Regions of interest (ROI) were drawn on each image that contained tumor and then multiplied by slice thickness to obtain the tumor volume. If the tumor was seen in several contiguous slices, then tumor volumes were added together. To avoid overestimation of tumor size, one half of the volume from the most dorsal and ventral images containing tumor were used in the volume analysis. Assuming a tumor density of 1 g/ml, tumor volumes (mm³) were converted to weight (g) for analysis.

Cytotoxic Specificity of HA-TXL In Vitro

The human ovarian carcinoma cell lines, NMP-1 and SKOV-3ip, were determined to be CD44(+) by flow cytometry (data not shown). Initial in vitro experiments were designed to establish whether uptake and subsequent cytotoxic effects of HA-TXL on these cell lines was CD44-specific. The results in Table I demonstrate that for both cell lines, pre-blocking of HA binding sites with free HA inhibited the ability of HA-TXL to reduce target cell survival. This result reflects the predominant role of receptor (CD44)-specific uptake, compared to non-specific pinocytosis, of HA-TXL; however, the latter route of uptake should still be operant, leading to some non-HA-inhibitable uptake by and cytotoxicity in CD44(+) cells, as well as with CD44(−) cells. These results are consistent with those of Luo and Prestwich who demonstrated CD44-specific uptake and internalization of fluorescently-labeled HA and cytotoxicity of HA-TXL against CD44(+) SKOV-3 and other tumor cells, whereas HA-TXL was ineffective against CD44(−) NIH3T3 target cells. Luo, Y., et al., A Hyaluronic Acid-Taxol Antitumor Bioconjugate Targeted to Cancer Cells, Biomacromolecules, 2000, 1(2):208-18. The relatively flat dose-response of cytotoxicity vs. HA-TXL concentration in these studies is reminiscent of the response to free Taxol that had previously been observed with NMP-1 and HEY ovarian carcinoma models, and in that light makes the observed extent of blockade with free HA more compelling.

TABLE I
Specificity of HA-TXL Cytotoxicity Against CD44(+)
Human Ovarian Carcinoma Cell Lines: Blocking by Free HA

Percent survival 4 hour HA-TXL Treatment)

| HA-TXL (ng/ml) | SKOV-3ip. | NMP-1 |
|---|---|---|
| 5000 | 55.9 ± 7.0^{a} | 67.6 ± 4.6 |
| +free HA^{b} | 104.8 ± 9.6^{c} | 86.5 ± 3.7 |
| 500 | 81.8 ± 14.5 | 73.0 ± 5.2 |
| +free HA | 101.9 ± 11.3 | 96.5 ± 4.1^{c} |
| 50 | 74.8 ± 12.3 | 78.7 ± 4.0 |
| +free HA | 91.6 ± 8.5^{c} | 79.3 ± 4.5 |

^{a}Mean ± SEM compared to untreated or HA-treated controls,
^{b}100-fold molar excess HA equivalents, pre-incubated for 4 hr prior to HA-TXL addition,
^{c}p < 0.03 (t-test) vs. HA-TXL without pre-blocking

Preliminary Toxicity Studies of HA-TXL

Mice were injected intraperitoneally with HA-TXL at doses up to 300 mg/kg (paclitaxel equivalents) and these mice were held for observation for at least six months. The mice were found to tolerate even the highest dose administered, indicating that this formulation was far less toxic than free paclitaxel (Taxol). Further, the 250 and 300 mg/kg doses exceeded the highest dose previously used (200 mg/kg) with another paclitaxel prodrug, poly(L-glutamic acid)-paclitaxel (PGA-TXL), suggesting HA-TXL might have an even higher mouse MTD than PGA-TXL. It is also considerably higher than the 100 mg/kg recently reported as the MTD for another hyaluronic acid-paclitaxel prodrug formulation, HYTAD1-p20. Rosato, A., et al., HYTAD1-p20: A New Paclitaxel-Hyaluronic Acid Hydrosoluble Bioconjugate for Treatment of Superficial Bladder Cancer, Urol Oncol, 2006, 24:207-215.

Antitumor Efficacy of HA-TXL

Both MR imaging-based anti-tumor effects and effects on survival following HA-TXL treatment in CD44(+) NMP-1 and SKOV-3ip orthotopic (intraperitoneal) xenograft models were evaluated.

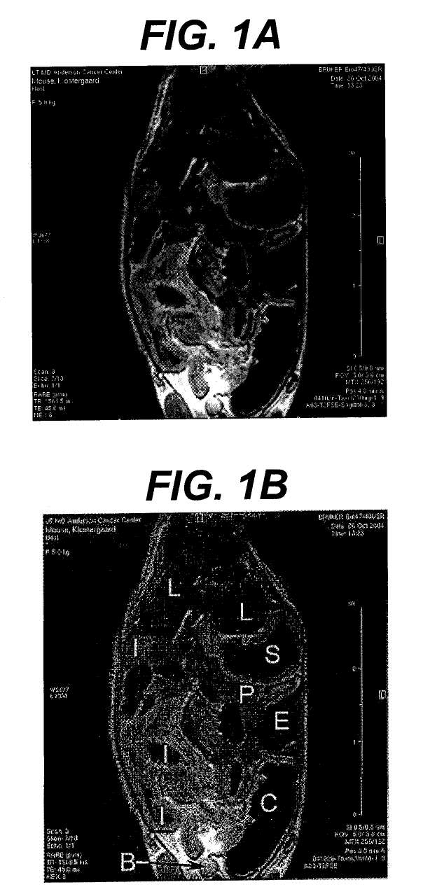

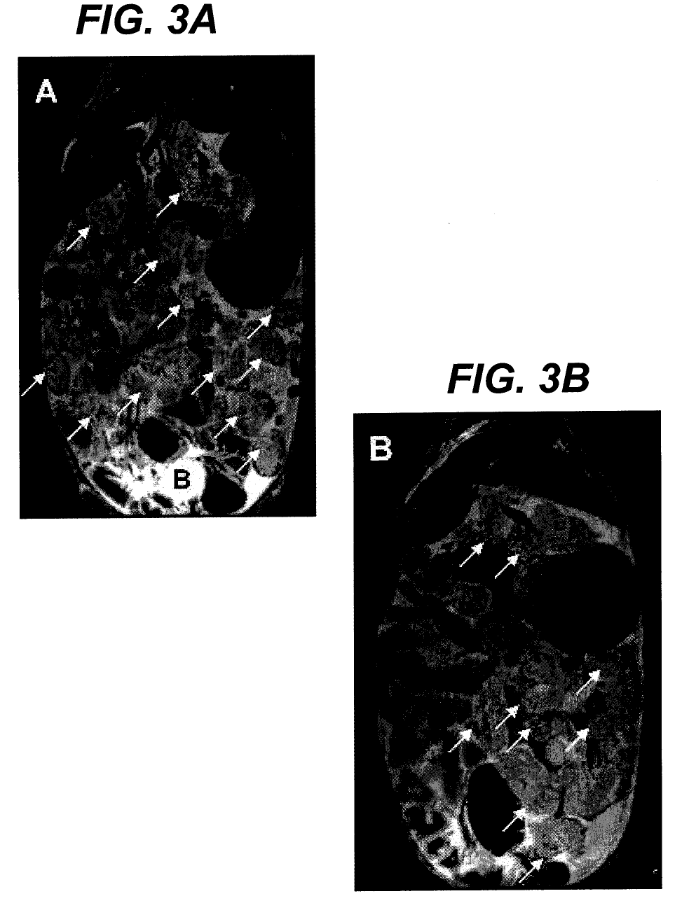

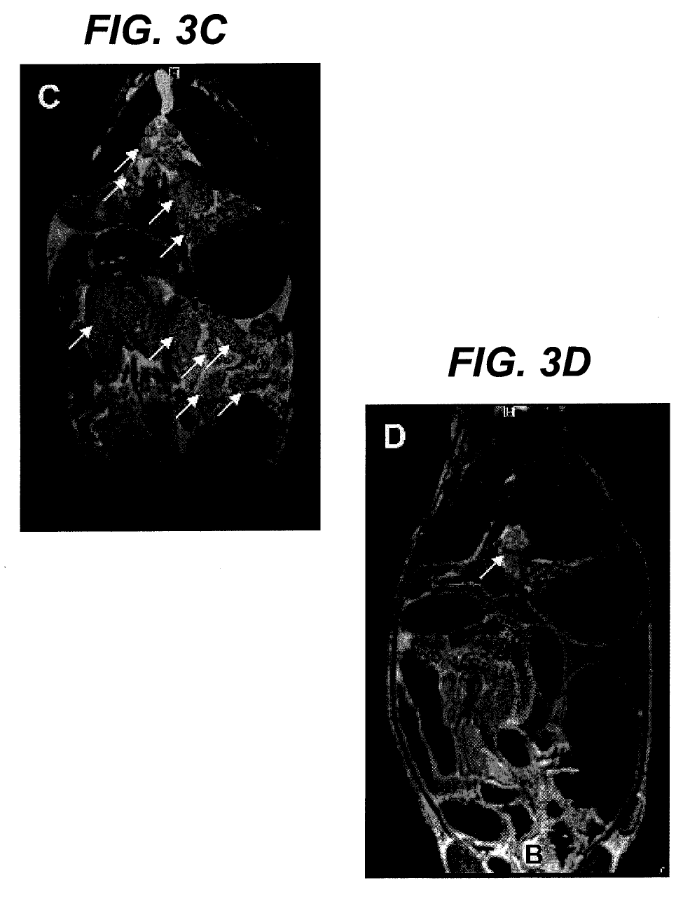

NMP-1: In a pilot efficacy experiment, mice bearing NMP-1 xenografts received an intraperitoneal injection of HA-TXL (100 or 200 mg/kg, paclitaxel equivalents) on Day 8 post-tumor implantation. The control mice survived for an average of 34 days, the 100 mg/kg HA-TXL-treated mouse survived to Day 60, and the 200 mg/kg HA-TXL-treated mouse was sacrificed on Day 199, and was judged tumor-free by MR imaging (FIGS. 1A and 1B.; compare to controls in FIG. 3A).

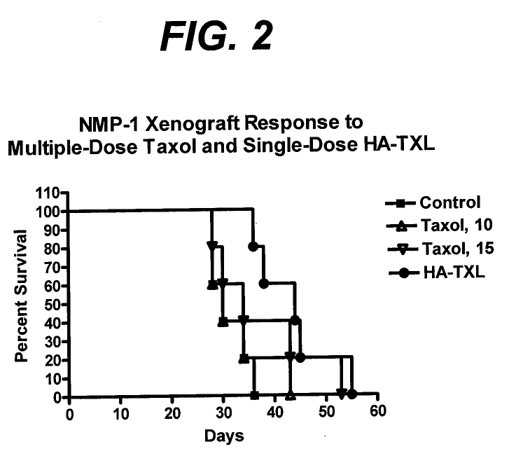

In an expanded efficacy experiment, groups of NMP-1-implanted mice were treated either with vehicle, with multiple dose regimens of Taxol, using 10 or 15 mg/kg (higher doses on this schedule are toxic), or with a single injection of HA-TXL. The effects on survival are shown in the Kaplan-Meyer survival plot in FIG. 2 and are summarized in Table II. In addition, two of five mice in each group were MR imaged on Day 28 post-tumor inoculation, prior to any mice requiring sacrifice. NMP-1-implanted mice responded to HA-TXL treatment with a T/C ˜140 (FIG. 2; p=0.004 by Mantel-Cox) and showed markedly reduced tumor burden (FIG. 3D) compared to controls (FIG. 3A). In contrast, multiple-dose regimens of Taxol at either dose level were essentially inactive in this model, both by MR imaging (FIG. 3B for 10 mg/kg and FIG. 3C for 15 mg/kg) and survival criteria (FIG. 2; T/C ˜105 for 10 mg/kg and ˜120 for 15 mg/kg).

TABLE II
Response of NMP-1 Xenograft Model to Multiple-
dose Taxol and Single-dose HA-TXL

| Treatment | | Mean Day of Survival/Sacrifice | T/C |
|---|---|---|---|
| Control | | 31.2 ± 3.2^{a} | — |
| Taxol | 10 mg/kg, | 32.6 ± 5.6 | 105 |
| | qd7 × 3^{b} | | |
| | 15 mg/kg, | 37.6 ± 9.3 | 120 |
| | qd7 × 3^{c} | | |
| HA-TXL | 180 mg/kg^{d} | 43.6 ± 6.7 | 140^{e} |

^{a}Mean ± SEM,
^{b}Taxol regimens initiated on Day 7 post-tumor inoculation,
^{c}Higher doses caused toxicity on this schedule,
^{d}Single dose administered on Day 7,
^{e}p = 0.004 vs. controls by Mantel Cox

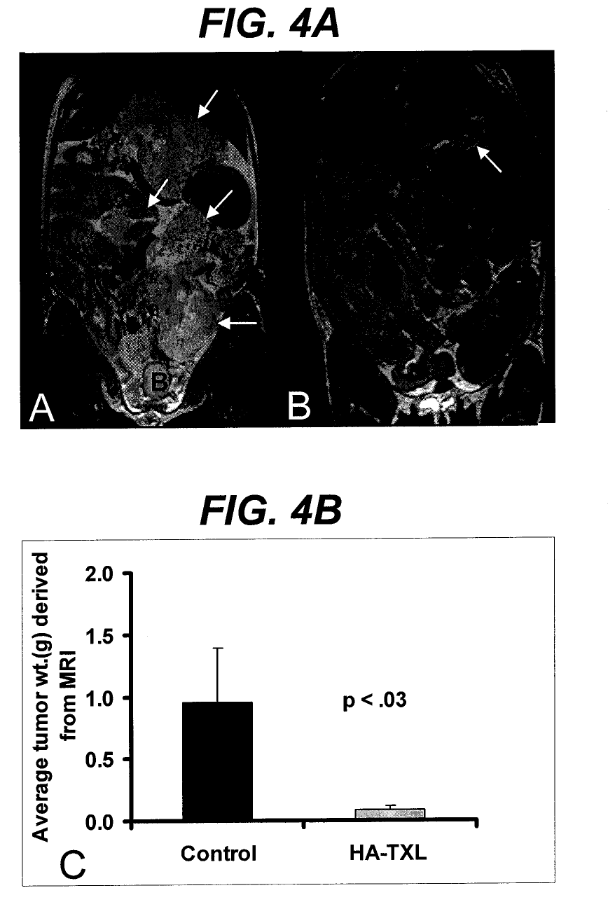

SKOV-3ip: Anti-tumor efficacy results with HA-TXL were generally similar to those with the SKOV-3ip ovarian carcinoma model. Necropsy examination conducted by a board-certified veterinary pathologist (REP) on the mice from the HA-TXL-treatment group found only small tumors, 12 weeks post-tumor implantation and 10 weeks post-treatment. However, the control SKOV-3ip mice all presented evidence for marked tumor involvement, typically including abdominal distention with bloody ascites and marked abdominal tumor burden associated with the umbilicus, diaphragm, abdominal wall, lymph nodes, and mesentery. MR images obtained on the day of sacrifice were analyzed by a diagnostic imaging clinician (VK) and representative images are shown in FIG. 4A; again, these images show clear distinctions between treated and control groups. Only small tumors were detected in HA-TXL-treated mice (Panel B), whereas significant tumor burden and resultant abdominal distention was very apparent in the control mice (Panel A). Quantification of contiguous MR images demonstrated that tumor burden in the HA-TXL-treated group was markedly reduced compared to controls (p<0.03, t-test; FIG. 4B).

Thus, in the SKOV-3ip model, both MR imaging and histopathological analyses support the anti-tumor efficacy of even a single dose of HA-TXL administered at a sub-MTD level.

Preliminary Toxicology Studies of HA-TXL

Aside from CD44, originally associated with lymphocyte activation, other HA receptors include RHAMM (receptor for HA-mediated cell motility) and HARLEC (HA receptor, liver endothelial cell). Thus, studies were conducted to determine whether as a result of expression of HARLEC or other HA receptors, HA-TXL treatment would be associated with significant hepatotoxicity. In preliminary studies, only slight elevation of serum liver transaminase (AST=220 U/ml, ALT=175 U/ml) and alkaline phosphatase (92 U/ml) levels 24 hr after intraperitoneal injection of 180 mg/kg HA-TXL was observed. It is possible that these toxicities were secondary to liver uptake, particularly the transaminase elevations; however, HARLEC and RHAMM are less specific for HA than is CD44 and the former can be blocked with chondroitin sulfate. Mahteme, H., et al., Uptake of Hyaluronan in Hepatic Metastases After Blocking of Liver Endothelial Cell Receptors, Glycoconj J., 1998, 15(9):935-939. This pre-blocking strategy should shunt HA-TXL away from certain normal tissues and increase uptake in tumor.

Certain studies have focused on CD44(+) human ovarian carcinoma models. The selectivity of HA-TXL for these CD44-expressing cell lines has been demonstrated in vitro by competition experiments with free HA (Table I); similar observations of CD44-specific uptake and cytotoxicity of HA-TXL have been reported previously, as well as lack of effects against CD44(−) NIH3T3 cells. Luo, Y., et al., Synthesis and Selective Cytotoxicity of a Hyaluronic Acid-Antitumor Bioconjugate, Bioconjug Chem, 1999, 10(5):755-63; Luo, Y., et al., A Hyaluronic Acid-Taxol Antitumor Bioconjugate Targeted to Cancer Cells, Biomacromolecules, 2000, 1(2):208-18. To further understand the nature of the HA/CD44 interaction and the role it might play in the selectivity of the response to HA-TXL in vivo, a control study using CD44(−) tumor models may be of interest. However, neither a CD44(−) human ovarian carcinoma model nor another CD44(−) tumor model with peritoneal metastases has been defined for such evaluation. Further, both potentially tumor-promoting and/or tumor-inhibiting effects of free HA in CD44(+) models must be properly controlled for in such analyses. Nevertheless, by employing a similar competition strategy with co-administered free HA, the relative roles of receptor-specific vs. pinocytotic uptake of HA-TXL in vivo with CD44(+) tumor models may be understood.

Other studies have begun to evaluate the anti-tumor efficacy of prodrug formulations based on an HA backbone or ligand. Klostergaard, J., et al., Magnetic Resonance Imaging-Based Prospective Detection of Intraperitoneal Human Ovarian Carcinoma Xenografts Treatment Response, Int J Gynecol Cancer, 2006, 16 Suppl 1:111-7; Rosato, A., et al., HYTAD1-p20: A New Paclitaxel-Hyaluronic Acid Hydrosoluble Bioconjugate for Treatment of Superficial Bladder Cancer, Urol Oncol, 2006, 24:207-215; Coradini, D., et al., Hyaluronic-Acid Butyric Esters as Promising Antineoplastic Agents in Human Lung Carcinoma: A Preclinical Study, Invest New Drugs, 2004, 22(3):207-17; Speranza, A., et al., Hyaluronic Acid Butyric Esters in Cancer Therapy, Anticancer Drugs, 2005, 16(4):373-9 Review; Peer, D., et al., Tumor-Targeted Hyaluronan Nanoliposomes Increase the Antitumor Activity of Liposomal Doxorubicin in Syngeneic and Human Xenograft Mouse Tumor Models, Neoplasia, 2004, 6(4):343-353. For example, butyric acid esters of HA were prepared and these conjugates were injected intratumorally in an s.c.-implanted syngeneic Lewis lung carcinoma model. The growth rate of the ectopic tumor was reduced compared to the vehicle control, and both the number and weight of lung metastases were significantly reduced compared to controls. Coradini, D., et al., Hyaluronic-Acid Butyric Esters as Promising Antineoplastic Agents in Human Lung Carcinoma: A Preclinical Study, Invest New Drugs, 2004, 22(3):207-17; Speranza, A., et al., Hyaluronic Acid Butyric Esters in Cancer Therapy, Anticancer Drugs, 2005, 16(4):373-9 Review. The previously reported studies did not involve the use of an orthotopic (intraperitoneal) human tumor xenograft or administration of the HA prodrug loco-regionally (intraperitoneal) rather than intratumorally. However, a different study as reported the use of an HA backbone for a paclitaxel prodrug (HYTAD1-p20). Rosato, A., et al., HYTAD1-p20: A New Paclitaxel-Hyaluronic Acid Hydrosoluble Bioconjugate for Treatment of Superficial Bladder Cancer, Urol Oncol, 2006, 24:207-215. In an ectopic human bladder carcinoma xenograft model in SCID mice, multiple-dose regimens of HYTAD1-p20 administered intraperitoneally or Taxol administered intravenously (i.v.) achieved comparable tumor growth inhibition. Nevertheless, results from an orthotopic NMP-1 model demonstrate superior anti-tumor efficacy with even a single dose of HA-TXL compared to a multiple-dose Taxol regimen.

Although HA may be viewed as simply a backbone by which paclitaxel (and other) chemotherapeutics might be delivered to CD44(+) tumor cells, the possibility that part of the anti-tumor effect of HA-TXL might be mediated by the backbone itself has not been ruled out. For example, HA may disrupt CD44(+) tumor cell-extracellular matrix interactions, presumably leading to anoikis, as has been observed in a human breast carcinoma xenograft model. Herrera-Gayol, A., et al., Effect of Hyaluronan on Xenotransplanted Breast Cancer, Exp Mol Pathol, 2002, 72:179-185. In that light, comparisons of HA-TXL anti-tumor efficacy against tumor models with even greater taxane-resistance can be helpful to distinguish direct effects on either the tumor or stromal compartments.

In view of the recent clinical trial results demonstrating the survival benefit of intraperitoneal (i.p.) vs. intravenous (i.v.) administration of chemotherapeutic agents for ovarian cancer patients with small volume peritoneal disease, some pre-clinical evaluations of HA-TXL have been confined to the intraperitoneal administration route. However, this does not exclude the possibility that the intravenous administration route would also demonstrate anti-tumor efficacy, although such direct exposure to CD44(+) leukocyte populations might have undesired effects on immune function; nor does it address the actual pharmacological behavior and mode of uptake of HA-TXL administered intraperitoneally. Although a reasonable model for the latter may be one involving direct uptake of HA-TXL from the peritoneum into the tumor milieu, one cannot currently exclude the possibility of clearance from the peritoneum, followed by systemic distribution and extravasation from the tumor vasculature in the small tumor foci present at the time of treatment. El-Kareh, A. W., et al., A Theoretical Model for Intraperitoneal Delivery of Cisplatin and the Effect of Hyperthermia on Drug Penetration Distance, Neoplasia, 2004, 6(2):117-127. Further, another setting in which HA-TXL-based therapy might have a sound rationale is in metronomic therapy, as the absence of polyoxyl 40 hydrogenated castor oil (Cremophor; Sigma-Aldrich, St. Louis, Mo.) would obviate the interference of this excipient with the anti-angiogenic effects of taxanes, and paclitaxel in particular. Metronomic therapy is generally discussed in Kamat et at, Metronomic Chemotherapy Enhances the Efficacy of Antivascular Therapy in Ovarian Cancer, CANCER RES. 2007; 67: (1). Jan. 1, 2007.

A number of variables which may be optimized include the size of the HA backbone, as this is thought to affect the rates of HA-TXL clearance from the peritoneum and from the vascular compartment, as well as the opportunity for multiple CD44/HA binding interactions, and hence the resultant avidity. Similarly, the extent of paclitaxel substitution in the current studies was intentionally kept at about 10% or less of the available carboxyl groups on the HA, with the expectation that this would have minimal effect on the HA/CD44 interactions. However, higher loading may be acceptable, particularly with longer HA chains that allow multiple receptor interactions.

### Example 2

The in vitro effect of an anti-cancer agent-hyaluronic acid conjugate of the present disclosure, HA-paclitaxel, on squamous cell carcinomas of the head and neck (SCCHN) cell lines was determined using a 3-(4,5-dimethylthiazol-2-yl)-2,5-diphenyltetrazolium bromide cell growth assay. The antitumor effects of HA-paclitaxel were assessed in orthotopic xenograft models of SCCHN. Treatment with HA-paclitaxel showed dose-dependent inhibition of cell growth which was blocked with free HA. HA-paclitaxel was tolerated at 120 mg/kg paclitaxel equivalents in the nude mouse model and i.v. administration of this compound significantly inhibited tumor growth in vivo. Animal survival was prolonged in a paclitaxel-sensitive cell line (OSC19-luciferase, IC₅₀ 2.16 nM), but not in a relatively paclitaxel-resistant cell line (HN5, IC₅₀ 4.58 nM). Tumor vasculature was significantly inhibited by treatment with HA-paclitaxel as compared to paclitaxel alone.

Measurement of Cell Proliferation

To test the ability of paclitaxel and HA-paclitaxel to inhibit the proliferation of all human squamous cancer cell lines in vitro, a 3-(4,5-dimethylthiazol-2-yl)-2,5-diphenyltetrazolium bromide (MTT) assay was used. Two thousand cells per well were grown in DMEM medium supplemented with 10% FBS in 96-well tissue culture plates. After 24 h, the cells were treated with various concentrations of paclitaxel or HA-paclitaxel in DMEM medium supplemented with 2% FBS. To measure the number of metabolically active cells after a 3-day incubation period, an MTT assay as measured by a 96-well microtiter plate reader (MR-5000; Dynatech Laboratories Inc, Chantilly, Va.) at an optical density of 570 nm was used.

Animals and Maintenance

Eight-to-12-week-old male athymic nude mice were purchased from the National Cancer Institute (Bethesda, Md.). The mice were kept in a specific pathogen-free facility and were fed irradiated mouse chow and autoclaved reverse osmosis-treated water. The housing and care of the mice were approved by the American Association for Accreditation of Laboratory Animal Care and met all current regulations and standards of the U.S. Department of Agriculture, U.S. Department of Health and Human Services, and the National Institutes of Health. Animal procedures were done according to a protocol approved by the Institutional Animal Care and Use Committee of The University of Texas M.D. Anderson Cancer Center.

Cell Lines

The OSC19-luciferase line was created in the laboratory of Jeffrey Myers, Md., Ph.D in the Department of Head and Neck Surgery at M. D. Anderson Cancer Center. The parental cell line was originally created by as described by Yokoi et al. Expression of luciferase was induced using a lentiviral vector containing firefly luciferase. The HN5 cell line was obtained from Dr. Luka Milas (MD Anderson Cancer Center, Houston, Tex.).

Cells were grown in vitro in Dulbecco's modified Eagle's medium (DMEM) supplemented with 10% fetal bovine serum (FBS), L-glutamine, sodium pyruvate, nonessential amino acids and a twofold vitamin solution (Life Technologies, Inc., Grand Island, N.Y.). Adherent monolayer cultures were maintained on plastic and incubated at 37° C. in 5% carbon dioxide and 95% air. The cultures were free of Mycoplasma species and were maintained for no longer than 12 weeks after recovery from frozen stocks.

Chemical Compounds

Hyaluronic acid (˜35 kDa) was provided by K₃ Corporation (Great Falls, Va.),1-Ethyl-3-[3V-(dimethylamino)propyl]carbodiimide(EDCI), diphenylphosphoryl chloride, adipic dihyrazide (ADH), succinic anhydride, N-hydroxysuccinimide (NHS), and triethyl-amine were purchased from Sigma-Aldrich Co. (Milwaukee, Wis.). Paclitaxel (Taxol®) was purchased from HandeTech Development Co. (Houston, Tex.).

Synthesis of HA-paclitaxel: The reported synthesis of Auzenne et al. was followed. Auzenne, E., et al., Superior Therapeutic Profile of Poly-L-glutamic Acid-Paclitaxel Copolymer Compared With Taxol in Xenogeneic Compartmental Models of Human Ovarian Carcinoma, Clin Cancer Res, 2002, 8(2): 573-81. HA-ADH (150 mg), prepared as described by Luo and Prestwich and Luo et al., was dissolved in 0.1M NaHCO₃ buffer (pH 8.5) at a concentration of 1 mg/ml. Luo, Y., et al., Synthesis and Selective Cytotoxicity of a Hyaluronic Acid-Antitumor Bioconjugate, Bioconjug Chem, 1999, 10(5):755-63; Luo, Y., et al., A Hyaluronic Acid-Taxol Antitumor Bioconjugate Targeted to Cancer Cells, Biomacromolecules, 2000, 1(2):208-18. To this solution was added paclitaxel-NHS ester (36 mg) dissolved in sufficient DMF-H₂O (2:1, vol/vol) to give a homogeneous solution. The reaction mixture was stirred at room temperature for 24 hours and then evaporated to dryness in vacuo (37 C). The residue was dissolved in H₂O, and the product was purified by gel filtration chromatography (Biogel P-10) using water as eluent. Fraction containing HA-paclitaxel, as evidenced by HPLC analysis, were combined and lyophilized. The percentage of incorporated paclitaxel was determined by UV absorbance.

Preparation of FITC-HA-Taxol: HA-paclitaxel (200 mg, with 7% paclitaxel loading) was dissolved in 0.1M NaHCO₃ buffer (15 ml, pH 8.5). FITC (15 mg, 39 μmol) in DMF (5 ml) was added to the reaction mixture and stirred overnight at room temperature. FITC-HA-paclitaxel was purified by dialysis against 50% acetone/H₂O. The purity was determined by HPLC.

Establishment of Orthotopic Nude Mouse Models of SCCHN and Therapy

OSC19-luc or HN5 cells were harvested from subconfluent cultures by trypsinization and washed. For all animal experiments, cells (100,000) were suspended in 30 μL of serum-free Dulbecco modified Eagle's medium (DMEM), and injected into the mouse tongue, as described previously. Seven days after the injection of OSC19-luc or HN5 cells, when tumors were already established, mice with similar tumor size as determined by tumor volume were randomized into four groups (10 mice per group): control, free paclitaxel, HA-paclitaxel, and HA alone. Drugs were administered intravenously by injection into the dorsal penile vein under loupe magnification. Animals were anesthetized for this procedure with pentobarbital as previously described. HA-paclitaxel was injected at 120 mg/kg paclitaxel equivalent and paclitaxel at 10 mg/kg in a total volume of 400 ul, near their multiple-dose MTDs. The control group received 400 ul sterile saline intravenously. An additional control group received an equivalent amount of free HA in a volume of 400 ul. Each animal received 3 weekly treatments.

The mice were examined twice a week for weight loss. The mice were euthanized by CO₂ asphyxiation at 60 days post-injection or earlier if they lost more than 20% of their pre-injection body weight or became moribund (indicated by a large tumor volume, hunched posture, and/or poor grooming). Tongue tumors were measured twice weekly with microcalipers and again at the time of sacrifice. Tumor volume (V) was calculated using the formula V=(A)(B²)π/6 (with A being the longest dimension of the tumor; and B being the dimension of the tumor perpendicular to A). The mice were necropsied, with removal of tongue tumors and cervical lymph nodes. Half of each tumor was fixed in formalin and embedded in paraffin for immunohistochemical analysis and hematoxylin and eosin (H&E) staining. The other half was embedded in optimal cutting temperature (OCT) compound (Miles, Inc., Elkhart, Ind.), rapidly frozen in liquid nitrogen, and stored at −80° C. The cervical lymph nodes were also embedded in paraffin and sectioned, stained with H&E, and evaluated for the presence of metastases.

Imaging of Orthotopic Tumors

Bioluminescence of the tongue tumors through standardized regions of interest was also quantified using Living Images (Xenogen, Alameda, Calif.). Seven days after orthotopic injections, animals with OSC-19-luc and JMAR-luc tumors were imaged on an approximately weekly basis. Animals were anesthetized by 2% isoflurane (Abbott, Abbott Park, Ill.) before and during imaging: mice were injected i.p. with luciferin (Xenogen) at 150 mg/kg in a volume of 0.1 mL {Jenkins, 2003 #7}. Animals were imaged at a peak time of 15 min post luciferin injection via a IVIS 200 Imaging System (Xenogen). The photons emitted from the luciferase-expressing cells within the animal were quantified using the software program Living Image as an overlay on Igor (Wavemetrics, Seattle, Wash.). Before use in vivo, engineered OSC-19-luc and JAM-luci cells were confirmed in vitro to homogeneously express high levels of luciferase as monitored by the IVIS imaging system.

Immunohistochemical Detection of CD31/Platelet/Endothelial Cell Adhesion Molecule 1

Frozen tissues were sectioned into 8- to 10-μm slices and used for detection of CD31/platelet/endothelial cell adhesion molecule 1 (CD31/PECAM). The slices were mounted on positively charged Plus slides (Fisher Scientific, Pittsburgh, Pa.) and air-dried for 30 minutes; fixed sequentially in cold acetone (5 minutes), 1:1 acetone/chloroform (v/v; 5 minutes), and acetone (5 minutes), and then washed with PBS. Immunohistochemical procedures were done as described previously with the primary antibody diluted 1:400. Peroxidase-conjugated secondary antibody was used for immunohistochemical analysis of CD31/PECAM. Bleaching of fluorescence was minimized by covering the slides with 90% glycerol and 10% PBS. The slides were incubated with stable 3,3′-diaminobenzidine for 10 to 20 minutes and then examined for the presence of CD31/PECAM. The sections were rinsed with distilled water, counterstained with Gill's hematoxylin for 1 minute, and mounted with Universal Mount (Research Genetics, Huntsville, Ala.).

Immunofluorescence

Immunofluorescence microscopy was done using a Nikon Microphot-FX equipped with a HBP 100 mercury lamp and narrow bandpass filters to individually select for green, red, and blue fluorescence (Chroma Technology Corp., Brattleboro, Vt.). Images were captured using a cooled CCD Hamamatsu 5810 camera (Hamamatsu Corp., Bridgewater, N.J.) and Optimas Image Analysis software (Media Cybernetics, Silver Spring, Md.). Photomontages were prepared using Adobe Photoshop software (Adobe Systems, Inc., San Jose, Calif.).

Quantification of Microvessel Density and Apoptotic Cells

To quantify microvessel density (MVD), areas containing higher numbers of tumor-associated blood vessels were identified at low microscopic power (100×). Vessels completely stained with anti-CD31 antibodies were counted in three random 0.04-mm² fields per slide at 200× magnification.

Quantification of apoptotic endothelial cells was expressed as the average of the ratios of apoptotic endothelial cells to the total number of endothelial cells in three random 0.04-mm² fields at 200× magnification.

Statistical Analysis

Best-fit curves were generated for the MTT and PI assays and used to determine the concentration at which 50% of the drug effect (IC₅₀) was exhibited. Quantified results of PCNA, CD31, and tumor volume were compared with Kruskal-Wallis and Wilcoxon rank-sum test, as appropriate. Survival was analyzed with the Kaplan-Meier method. Differences between the treatment and control groups were compared with the log-rank test. A two-tailed p<0.05 was considered significant.

HA-Paclitaxel Exerts Growth Inhibitory Effects In Vitro

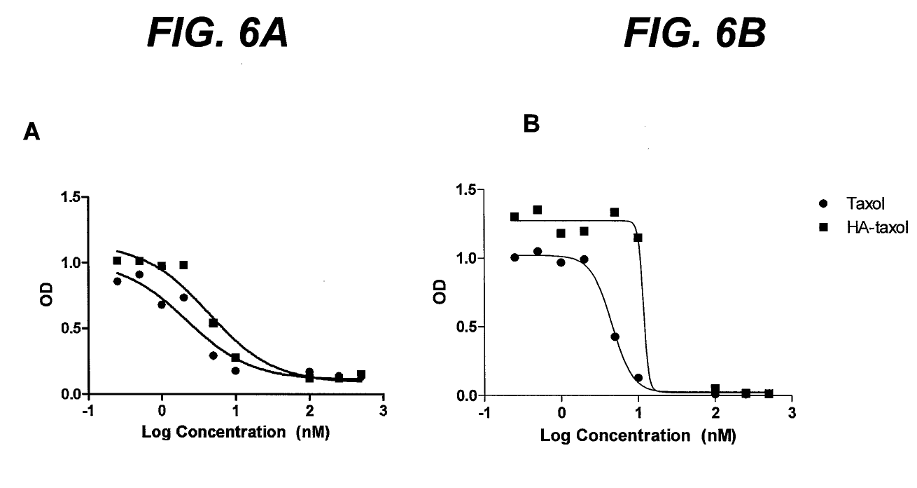

The in vitro effects of HA-paclitaxel were examined using the MTT assay. HA-paclitaxel showed significant growth inhibitory effects, but with slightly decreased potency as compared to paclitaxel alone for the OSC19-luciferase cell line (IC₅₀ 4.31 nM versus 2.16 nM, FIG. 6A). In the paclitaxel-resistant cell line, HN5, HA-paclitaxel was growth inhibitory at nanomolar concentration (IC₅₀ 11.77 nM), but had decreased potency as compared to paclitaxel (IC₅₀ 4.58 nM, FIG. 6B).

HA-Paclitaxel Growth Inhibition is Mediated Via Hyaluronic Acid Binding

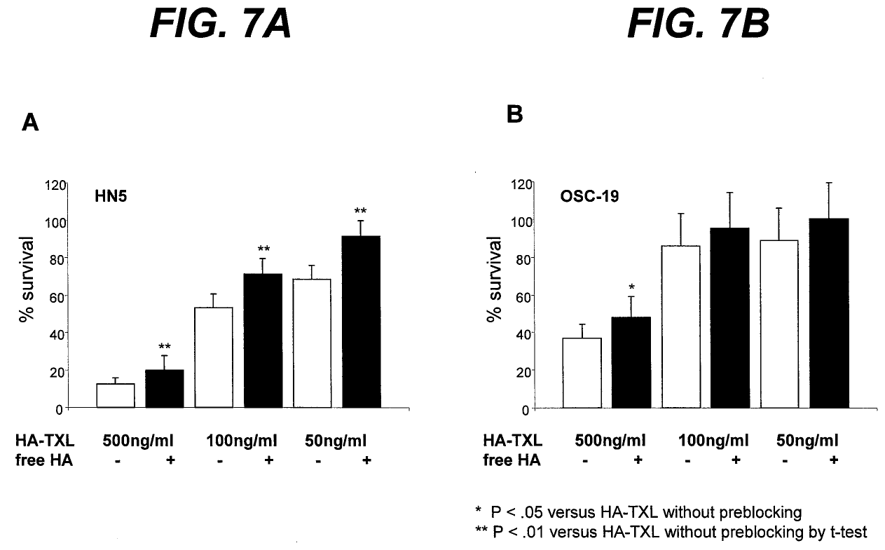

Blocking experiments were performed to determine the importance of HA binding to the internalization and growth inhibitory effects of HA-paclitaxel. For both cell lines, pre-incubation with excess free HA blocked the decrease in cell proliferation induced by HA-paclitaxel (FIG. 7). This effect was significant in the HN5 cell line at all concentrations (p<0.01, FIG. 7A). In the OSC19-luciferase cell line, blocking was only demonstrated at 500 ng/ml HA-paclitaxel, but not at 100 or 50 ng/ml (FIG. 7B).

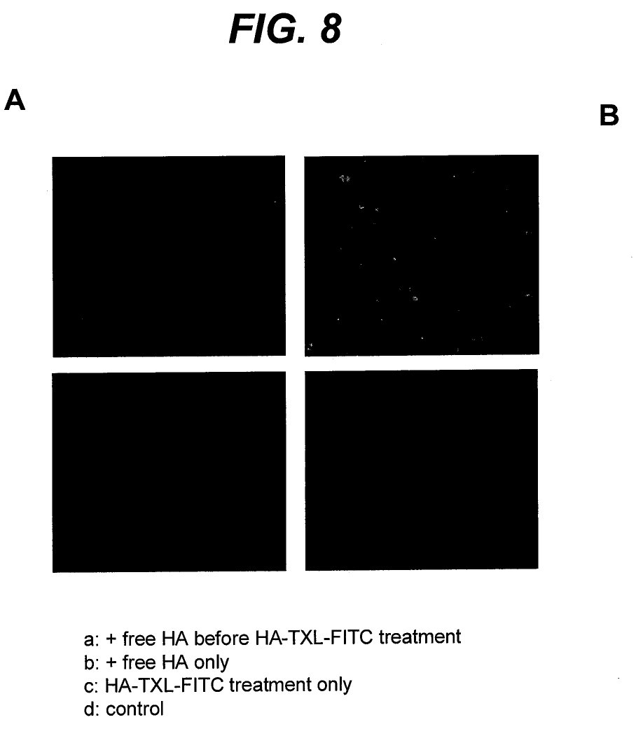

An additional experiment was performed to visualize uptake of HA-paclitaxel-FITC in vitro. Pre-blocking of HA binding sites with free HA resulted in inhibition of uptake of HA-paclitaxel-FITC. As shown in FIG. 8A, HA-paclitaxel-FITC can be seen within the cytoplasm of untreated cells, but not in cells pre-incubated with free HA. Quantitatively, incubation with HA significantly decreased the uptake of HA-paclitaxel-FITC (P<0.01, FIG. 8B).

Treatment with HA-Paclitaxel Inhibits In Vivo Growth of Oral Tongue Tumor Xenografts in an Orthotopic Nude Mouse Model.

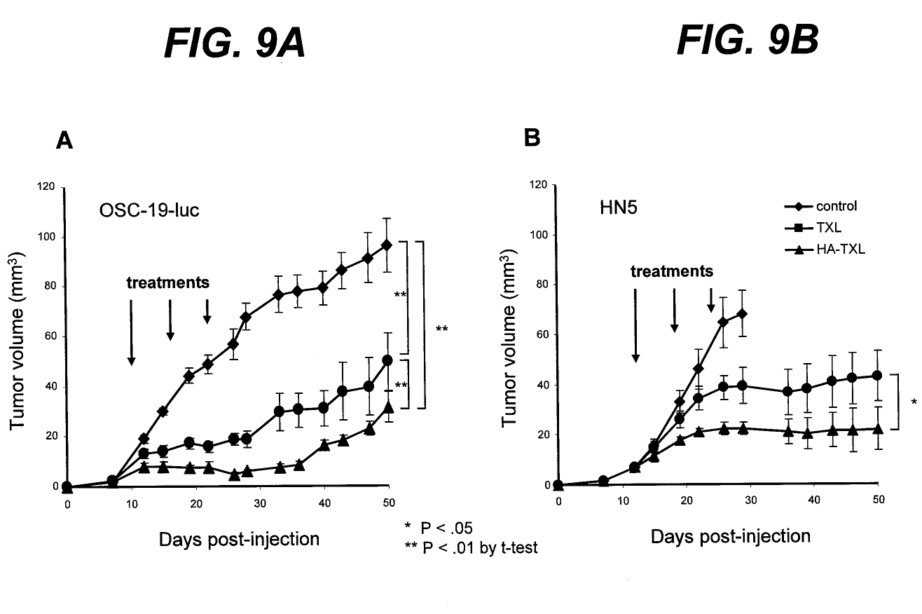

The anti-tumor efficacy of HA-paclitaxel in xenograft models of oral tongue SCC was assessed using three groups: control, intravenous free paclitaxel, and intravenous HA-paclitaxel. Cells were injected as described and tumors assessed by visual inspection and bioluminescence prior to randomization. Three weekly treatments were administered and tumor growth monitored for 7 weeks. Treatment with free paclitaxel decreased the growth of tumor in OSC19 by 64.2% whereas HA-paclitaxel reduced tumor growth by 90.7% one week after the last treatment (p<0.01, FIG. 9A). A group receiving intravenous free HA alone showed no significant difference as compared to control (data not shown).

Similar inhibition of tumor growth was observed using the HN5 model, with growth reduction of 63.8% with paclitaxel and 86.2% with HA-paclitaxel (p<0.01, FIG. 9B). In both cases, there was a statistically significant decrease in tumor growth for HA-paclitaxel treatment as opposed to treatment with free paclitaxel (p<0.01 OSC19-luciferase, p<0.05 HN5). Interestingly, HN5 xenografts displayed minimal tumor growth after the cessation of treatment whereas the OSC19-luciferase xenografts demonstrated resumption of tumor growth after approximately 20 days of stasis.

Reduction of Bioluminescence in Orthotopic Tumor Xenograft

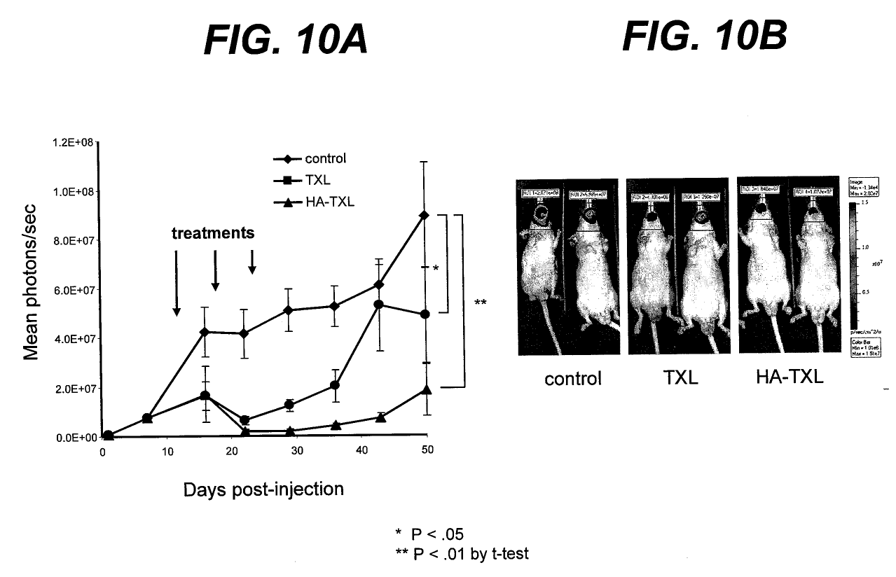

OSC19-luciferase is a modified cell line expressing the firefly luciferase protein and enabling measurement of bioluminescence in living animals as an estimation of viable tumor. It was found that treatment with either HA-paclitaxel or free paclitaxel caused a significant decrease in bioluminescence (FIGS. 10A and 10B). Bioluminescence was reduced by 99.2% in the HA-paclitaxel treated animals and by 86.5% in paclitaxel treated animals as opposed to control (p<0.01) as measured at one week after the last treatment. The HA-paclitaxel treated group had significantly lower bioluminescence compared to the free paclitaxel treated group (p<0.01).

Treatment with HA-Paclitaxel Prolongs Survival in an Orthotopic Nude Mouse Model of HNSCC

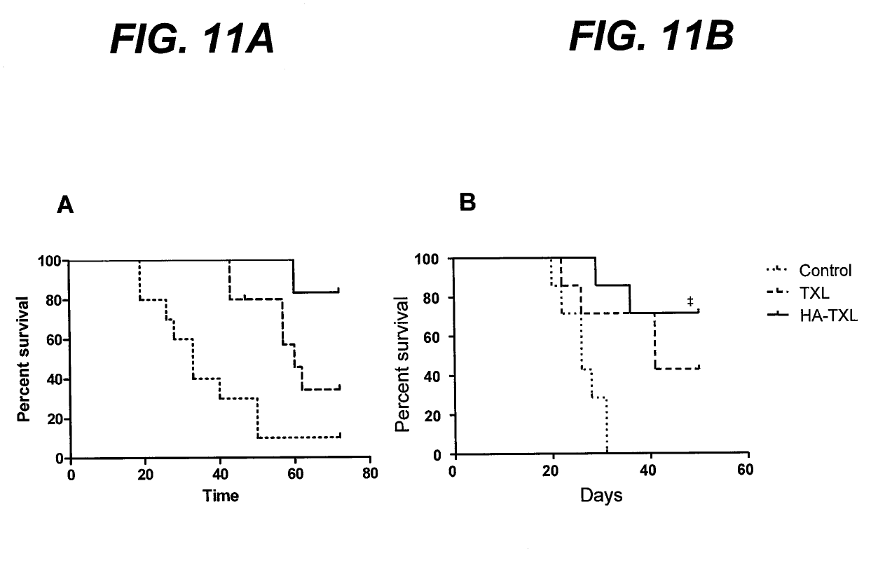

After completion of three weekly injections of control, paclitaxel, or HA-paclitaxel, animals were followed until they met criteria for sacrifice as previously described. Treatment with HA-paclitaxel or free paclitaxel resulted in increased survival for both tumor models as compared to control by log-rank test (p<0.001, FIG. 11A). Median survival time for control, paclitaxel, and HA-paclitaxel was 30, 60, and 79 days for OSC19-luciferase and 26, 40, and 45 days for HN5. On comparison between groups, treatment with HA-paclitaxel improved survival as compared to paclitaxel for OSC19-luciferase (FIG. 11A), but no significant difference was seen with HN5 (FIG. 11B).

HA-Paclitaxel Treatment Inhibits Angiogenesis In Vivo

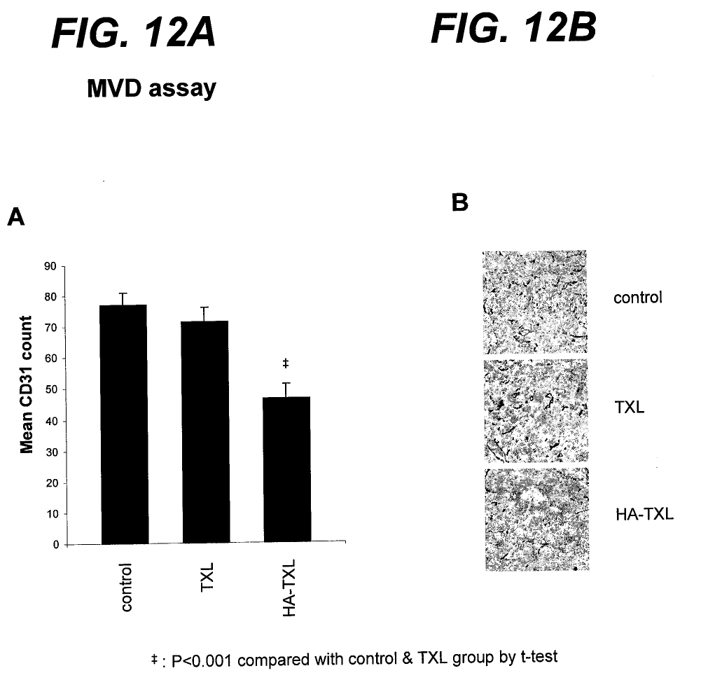

Frozen tissue sections from animals treated with weekly injections of control, paclitaxel and HA-paclitaxel (as described above) were examined for CD31 staining as a measure of angiogenesis (FIGS. 12A and 12B). Treatment with free paclitaxel had no effect on MVD, whereas treatment with HA-paclitaxel significantly reduced MVD (p<0.001).

Results

The findings above indicate that HA-paclitaxel exhibits cytotoxic effects on HNSCC cell lines in vitro and reduced tumor volume and prolonged survival in orthotopic HNSCC nude mouse xenograft models. HA-paclitaxel had slightly less potency in vitro than paclitaxel alone, but remained inhibitory at nanomolar concentrations. Entry of HA-paclitaxel into cells and downstream reduction in cell proliferation were partially blocked by free HA. It was also shown that three weekly injections of HA-paclitaxel were more effective than paclitaxel alone in inhibiting growth of tumors in an animal model. HA-paclitaxel, but not paclitaxel alone, also resulted in a delay in further tumor growth in HNSCC models for several weeks after the cessation of treatment. HA-paclitaxel was tolerated at high paclitaxel equivalent doses when injected intravenously and caused decreased microvessel density in tumor specimens.

The findings also showed the efficacy and safety of intravenous administration of HA-paclitaxel. The paclitaxel equivalent dosage used in the experiments was 12 times higher than the MTD of intravenous paclitaxel determined for our mouse mode, with no evidence of increased toxicity (data not shown). Further increases in dose were not attempted due to solubility and volume issues with intravenous injection in mice, but previous data found no toxicity with intraperitoneal injection of up to 300 mg/kg dose equivalent. No prior studies have used the intravenous route of administration of HA-paclitaxel, although several clinical trials have been performed with PGA-paclitaxel injected intravenously to treat advanced solid tumors; no significant toxicity has been noted in studies with biopolymer conjugates in animal models or in patients. Conjugation of paclitaxel appears therefore to offer a therapeutic advantage over unmodified paclitaxel.

The data herein demonstrates that HA-paclitaxel more effectively inhibits growth of HNSCC xenografts and improves survival when compared to unmodified paclitaxel. It is believed that this increase is likely due to the increased amount of drug that can be given as well as the more favorable pharmacokinetics of conjugated paclitaxel. Furthermore, HA-paclitaxel exhibited a static effect in terms of tumor growth that was persistent after cessation of therapy, an effect rarely seen on tumor growth with other agents in our models.

Conjugated paclitaxel has significantly increased half-life in plasma whether injected intraperitoneally or intravenously in pharmacokinetic studies. Data from Banzato et al. showed HA-paclitaxel to be persistently elevated in the plasma for 120 hours after IP administration; AUC was 144 μg h/mL for paclitaxel and 1,069 μg h/mL for HA-paclitaxel. A pharmacokinetic study of PGA-paclitaxel injected intravenously showed a comparable increase in elimination half-life for the conjugated drug (108-261.5 hours) as well as a further increase in AUC (1-2% for unmodified paclitaxel as compared to the study drug). Although the exact pharmacokinetic parameters for HA-paclitaxel injected intravenously have not been documented, data from IP and IV administration of similar conjugated agents such as PPX suggest that prolonged plasma concentration and exposure of the tumor to paclitaxel are a probable mechanism for the efficacy of this approach. While the peak of paclitaxel is not as high for conjugate compounds, the continued presence of low levels of paclitaxel may be exerting anti-angiogenic effects as seen with metronomic chemotherapeutic dosing.

### REFERENCES

1. Luo, Y., et al., Synthesis and Selective Cytotoxicity of a Hyaluronic Acid-Antitumor Bioconjugate, Bioconjug Chem, 1999, 10(5):755-63.
2. Luo, Y., et al., A Hyaluronic Acid-Taxol Antitumor Bioconjugate Targeted to Cancer Cells, Biomacromolecules, 2000, 1(2):208-18.
3. Li, C., et al., Complete Regression of Well-Established Tumors Using a Novel Water-Soluble Poly(L-glutamic acid)-paclitaxel Conjugate, Cancer Res, 1998, 58(11):2404-9.
4. Li, C., et al., Antitumor Activity of Poly(L-glutamic acid)-paclitaxel on Syngeneic and Xenografted Tumors, Clin Cancer Res, 1999, 5(4):891-7.
5. Li, C., et al., Biodistribution of Paclitaxel and Poly(L-glutamic acid)-paclitaxel Conjugate in Mice With Ovarian OCa-1 Tumor, Cancer Chemother Pharmacol, 2000, 46(5):416-22.
6. Zou, Y., et al., Effectiveness of Water Soluble Poly(L-glutamic acid)-camptothecin Conjugate Against Resistant Human Lung Cancer Xenografted in Nude Mice, Int J Oncol, 2001, 18(2):331-6.
7. Auzenne, E., et al., Superior Therapeutic Profile of Poly-L-glutamic Acid-Paclitaxel Copolymer Compared With Taxol in Xenogeneic Compartmental Models of Human Ovarian Carcinoma, Clin Cancer Res, 2002, 8(2): 573-81.
8. Zou, Y., et al., Antitumor Activity of Hydrophilic Paclitaxel Copolymer Prodrug Using Locoregional Delivery in Human Orthotopic Non-Small Cell Lung Cancer Xenograft Models, Clin Cancer Res, 2004 10(21):7382-91.
9. Phase II Clinical Trial of XYOTAX in Non-Small Cell Lung Cancer to Continue, Expert Rev Anticancer Ther, 2002, 2(3):244-5.
10. Singer, J. W., et al., Garzone Poly-(L)-glutamic Acid-Paclitaxel (CT-2103) [XYOTAX], a Biodegradable Polymeric Drug Conjugate: Characterization, Preclinical Pharmacology, and Preliminary Clinical Data, Adv Exp Med Biol, 2003, 519:81-99 Review.
11. Langer, C. J., Dilemmas in Management: The Controversial Role of Chemotherapy in PS
2 Advanced NSCLC and the Potential Role of CT-2103 (Xyotax), Oncologist, 2004, 9(4):398-405 Review.
12. Boddy, A. V., A Phase I and Pharmacokinetic Study of Paclitaxel Poliglumex (XYOTAX), Investigating Both 3-Weekly and 2-Weekly Schedules, Clin Cancer Res, 2005, 11(21):7834-40.
13. Dipetrillo, T., et al., Paclitaxel Poliglumex (PPX-Xyotax) and Concurrent Radiation For Esophageal and Gastric Cancer: A Phase I Study, Am J Clin Oncol, 2006, 29(4):376-9.
14. Albain, K. S., et al., PIONEER: A Phase III Randomized Trial of Paclitaxel Poliglumex Versus Paclitaxel in Chemotherapy-Naive Women With Advanced-Stage Non-Small-Cell Lung Cancer and Performance Status of 2, Clin Lung Cancer, 2006, 7(6):417-9.
15. Fields, M. M., et al., Screening for Disease: Making Evidence-Based Choices, Clin J Oncol Nurs., 2006 Feb. 10, (1):73-6 Review.
16. Morrison, J., Advances in the Understanding and Treatment of Ovarian Cancer, J Br Menopause Soc., 2005 Jun. 11, (2):66-71 Review.
17. Parazzini, F., et al., Risk Factors for Different Histological Types of Ovarian Cancer, Int J Gynecol Cancer, 2004, 14(3):431-6.
18. Kringen, P., et al., TP53 Mutations in Ovarian Carcinomas From Sporadic Cases and Carriers of Two Distinct BRCA1 Founder Mutations; Relation to Age at Diagnosis and Survival, BMC Cancer, 2005, 5:134.
19. Greimel, E. R., et al., Randomized Study of the Arbeitsgemeinschaft Gynaekologische Onkologie Ovarian Cancer Study Group Comparing Quality of Life in Patients With Ovarian Cancer Treated With Cisplatin/Paclitaxel Versus Carboplatin/Paclitaxel, J Clin Oncol., 2006, 24(4):579-86.
20. Fields, M. M., et al., Screening for Disease: Making Evidence-Based Choices, Clin J Oncol Nurs., 2006 Feb. 10, (1):73-6 Review.
21. Parazzini, F., et al., Risk Factors for Different Histological Types of Ovarian Cancer, Int J Gynecol Cancer, 2004, 14(3):431-6.
22. Zhao, C., et al., Circulating Haptoglobin Is an Independent Prognostic Factor in the Sera of Patients With Epithelial Ovarian Cancer, Neoplasia, 2007, 9(1): 1-7.

We claim:

1. A method of making an anti-cancer agent-hyaluronic acid conjugate comprising coupling an anti-cancer agent with a hyaluronic acid at a pH between about 7.5 to 9.0.

2. The method of claim 1 wherein the anti-cancer agent comprises at least one N-hydroxysuccinimide ester of a taxane.

3. The method of claim 1 wherein the anti-cancer agent comprises at least one taxane.

4. The method of claim 1 wherein the anti-cancer agent comprises at least one taxane selected from the group consisting of paclitaxel, docetaxel, and a derivative thereof.

5. The method of claim 1 wherein the hyaluronic acid comprises adipic dihydrazido functionalized hyaluronic acid.

6. The method of claim 1 wherein the pH is maintained at a pH between about 7.5 to 9.0 through the use of a buffer system.

7. An anti-cancer agent-hyaluronic acid conjugate comprising an anti-cancer agent and a hyaluronic acid comprising more than one disaccharide unit, wherein the anti-cancer agent is conjugated to less than 10 percent of the disaccharide units of the hyaluronic acid.

8. The conjugate of claim 7 wherein the conjugate was made by combining an N-hydroxysuccinimide ester of a taxane and a hyaluronic acid at a pH between about 7.5 to 9.0

9. The conjugate of claim 7 wherein the anti-cancer agent comprises a N-hydroxysuccinimide ester of a taxane.

10. The conjugate of claim 7 wherein the anti-cancer agent comprises at least one taxane selected from the group consisting of paclitaxel, docetaxel, and a derivative thereof.

11. The conjugate of claim 7 wherein the hyaluronic acid comprises adipic dihydrazido functionalized hyaluronic acid.

12. An anti-tumor hyaluronic acid-based prodrug formulation comprising a single intraperitoneal administration of a sub-maximum tolerated dose of an anti-cancer agent-hyaluronic acid conjugate comprising an anti-cancer agent and a hyaluronic acid comprising more than one disaccharide unit, wherein the anti-cancer agent is conjugated to less than 10 percent of the disaccharide units of the hyaluronic acid.

13. A method of determining CD44 receptor selectivity of a prodrug comprising administering to a subject an anti-cancer agent-hyaluronic prodrug in combination with free hyaluronic acid.

14. A method of reducing or eliminating tumor growth rate in a subject in need thereof comprising administering a therapeutically effective amount of an anti-cancer agent-hyaluronic acid conjugate to the subject, wherein the conjugate comprises an anti-cancer agent and a hyaluronic acid comprising more than one disaccharide unit, and wherein the anti-cancer agent is conjugated to less than 10 percent of the disaccharide units of the hyaluronic acid.

15. A mixture comprising at least 10 percent of an anti-cancer agent-hyaluronic acid conjugate wherein said mixture was made by combining an N-hydroxysuccinimide ester of a taxane and a hyaluronic acid at a pH between about 7.5 to 9.0.

16. The mixture of claim 15 wherein the N-hydroxysuccinimide ester of a taxane comprises a paclitaxel-N-hydroxysuccinimide ester.

17. The mixture of claim 15 wherein the hyaluronic acid comprises adipic dihydrazido functionalized hyaluronic acid.

18. The method of claim 15 wherein the pH is maintained at a pH between about 7.5 to 9.0 through the use of a buffer system.
